(19) United States
(12) Patent Application Publication
(10) Pub. No.: US 2026/0335631 A1
(43) Pub. Date: Oct. 29, 2026

(54) SMOOTH SWITCHING BETWEEN MEDICAL DEVICE SETTINGS

(71) Applicant: Cochlear Limited, Macquarie University, NSW (AU)

(72) Inventors: Sara Ingrid Duran, Denver, CO (US); Lakshmish Ramanna, Parker, CO (US); Christopher Joseph Long, Centennial, CO (US)

(21) Appl. No.: 19/107,824

(22) PCT Filed: Sep. 1, 2023
(86) PCT No.: PCT/IB2023/058681

§ 371 Date: Feb. 28, 2025

Related U.S. Application Data
(60) Provisional application No. 63/404,709, filed on Sep. 8, 2022.

(51) Int. Cl. A61N 1/05; A61N 1/36; A61N 1/372

(52) U.S. Cl. CPC A61N 1/0541; A61N 1/36039; A61N 1/37235

(57) ABSTRACT

Presented herein are techniques for smooth switching between settings (e.g., processing settings, stimulation strategies, etc.) of a medical device. In particular, the techniques presented herein incrementally adjust operation of the medical device to switch between different settings in a manner that mitigates perceptual disruption to the recipient.

### BACKGROUND

### Field of the Invention

The present invention relates generally to smooth switching between settings of a medical device.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: receiving sound signals at a hearing device; delivering, based on the sound signals, stimulation signals to a recipient of the hearing device using first operational settings; determining that the stimulation signals are to be delivered to the recipient using second operational settings that are different from the first operational settings; and incrementally adjusting one or more parameters of the stimulation signals to transition from the first operational settings to the second operational settings.

In another aspect, one or more non-transitory computer readable storage media is provided. The one or more non-transitory computer readable storage media comprises instructions that, when executed by a processor, cause the processor to: receive input signals at a medical device; convert, using a first set of operational settings, the input signals to stimulation signals for delivery to a recipient of the medical device; determine to switch to a second set of operational settings; and gradually transition from use of the first set of stimulation signals to use of the second set of operational settings by incrementally adjusting parameters used to deliver the stimulation signals to the recipient.

In another aspect, a medical device is provided. The medical device comprises: one or more input elements configured to receive input signals; a processing path configured to convert the input signals into one or more output signals for delivery to a recipient of the medical device using a first set of operational settings; and a stimulus adaption and smoothing module configured to gradually adjust, over a period of time, operation of the processing path from use of the first set of operational settings to use of a second set of operational settings.

In another aspect, a hearing device is provided. The hearing device comprises: one or more microphones configured to receive sound signals; one or more processors configured to convert the sound signals into first processed output signals using a first set of sound processing settings; wherein the processor is configured to subsequently determine that the sound signals are to be processed using a second set of sound processing settings that are different from the first operational settings and convert the sound signals into second processed output signals using a first set of sound processing settings, and wherein the one or more processors are configured incrementally adjusting one or more processing to transition from the first operational settings to the second operational settings.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a graph illustrating various phases of an idealized action potential as the potential passes through a nerve cell;

FIG. 3 is a functional block diagram illustrating a hearing device in accordance with embodiments presented herein;

FIGS. 4A, 4B, 4C, 4D, and 4E are schematic diagrams illustrating the spatial resolution of electrical stimulation signals in accordance with embodiments presented herein;

FIG. 5 is a diagram illustrating transitioning from first sound settings to second sound settings using transitional sound settings in accordance with embodiments presented herein;

FIGS. 6A, 6B, 6C, 6D, and 6E are diagrams illustrating adjusting parameters to gradually switch from first sound settings to second sound settings in accordance with embodiments presented herein;

FIG. 7 is a flowchart of a method in accordance with embodiments presented herein;

FIG. 8 is a flowchart of another method in accordance with embodiments presented herein;

FIG. 9 is a functional block diagram of an implantable stimulator system with which aspects of the techniques presented herein can be implemented; and

FIG. 10 is a schematic diagram illustrating a vestibular stimulator system with which aspects of the techniques presented herein can be implemented.

### DETAILED DESCRIPTION

Presented herein are techniques for smooth switching between settings (e.g., processing settings, stimulation strategies, etc.) of a medical device. In particular, the techniques presented herein incrementally adjust operation of the medical device to switch between different settings in a manner that mitigates perceptual disruption to the recipient.

Certain aspects of the techniques presented herein can be implemented with a hearing device, where the operational settings can be adjusted in response to a change in an acoustic environment of the recipient. For example, in first acoustic environment, a first set of operational settings (e.g., generating stimulation signals with a first set of stimulation parameters/attributes) can be used to deliver stimulation signals to the recipient, where the first set of operational settings provide for optimal sound perception in the first acoustic environment. However, the first set of operational settings may be sub-optimal in a second acoustic environment and, as such, a second set of operational settings (e.g., generating stimulation signals with a second set of stimulation parameters/attributes) can be used to deliver stimulation signals to the recipient in the second acoustic environment. Directly switching from the first set of operational settings to the second set of operational settings can cause an abrupt change to the stimulation signal parameters/attributes that, in turn, can result in a perceptual (e.g., noticeable) disruption to the recipient. However, gradually adjusting parameters/attributes of the stimulation signals while switching/transitioning from the first set of operational settings to the second set of operational settings can provide a smooth transition and mitigate the perceptual disruption.

In operation, the switching from the first set of operational settings to the second set of operational settings (e.g., the adjusting of parameters/attributes of the delivered stimulation signals) can utilize a series of “transitional” or “intermediary” sets of operational settings. Each of the transitional/intermediary sets of operational settings are associated with different stimulation parameters/attributes that are between the stimulation parameters/attributes associated with the first set of operational settings and the stimulation parameters/attributes associated with the second set of operational settings.

In certain aspects, a number of different stimulation parameters/attributes can be adjusted to transition from a first set of operational settings to a second set of operational settings, and these parameters can be adjusted in a transitional or stepwise manner. In addition, the parameters can be adjusted in a number of different ways to provide the smooth transition, where the changes can be predetermined to set dynamically based on, for example, the first and second operation settings, an ambient (e.g., sound) environment, device status, etc.

In one embodiment, a transition between operational settings can be smoothed by incrementally adjusting one or more of a threshold level, a comfort level, and/or a volume of the stimulation signals delivered to the recipient. In another embodiment, a transition between operational settings can be smoothed by incrementally adjusting a degree of focusing of the stimulation signals (e.g., incrementally adjusting weights of the electrodes that comprise a channel along a continuum from the first set of operational settings to the second set of operational settings). In another embodiment, a number of channels stimulated in different electrode configurations can be gradually adjusted during the transition. By gradually and/or incrementally adjusting these and/or other parameters, a perceptual disruption can be mitigated when switching between the first operational settings and the second operational settings.

Merely for ease of description, the techniques presented herein are primarily described with reference to a specific implantable medical device system, namely a cochlear implant system. However, it is to be appreciated that the techniques presented herein can also be partially or fully implemented by other types of implantable or non-implantable medical devices. For example, the techniques presented herein can be implemented by other hearing device systems that include one or more other types of hearing devices, such as hearing aids, middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. In certain aspects, the techniques are generally applicable to a variety of sensory prostheses (e.g., hearing devices, vestibular implants, visual devices, etc.)

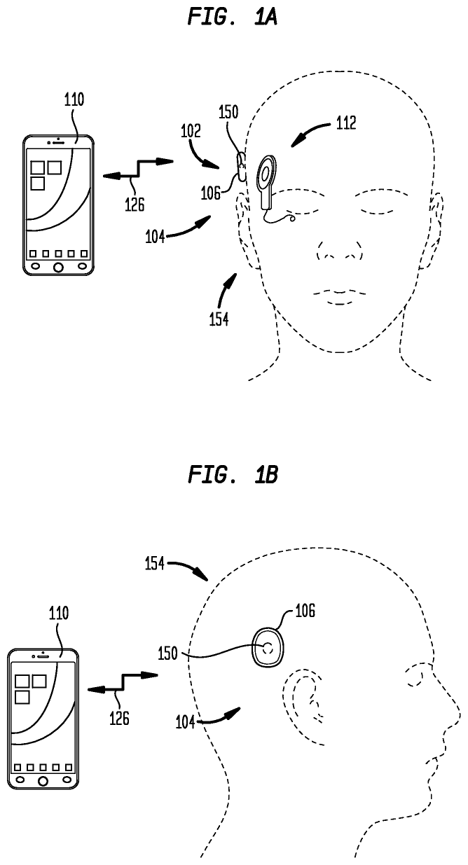

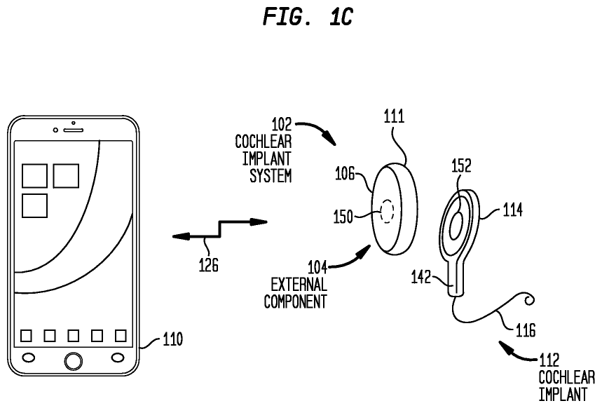

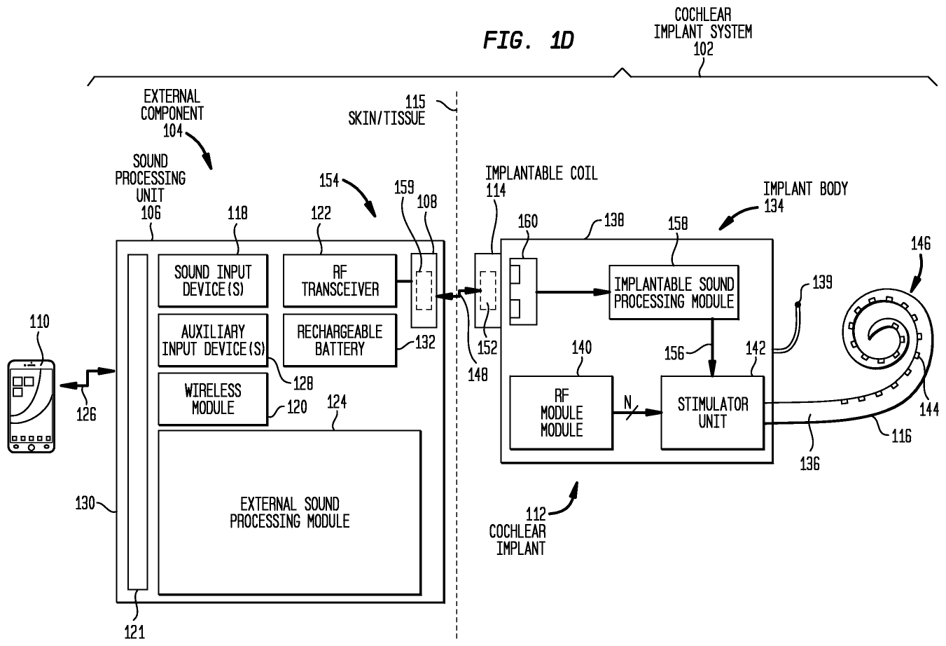

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient. In accordance with embodiments presented herein, the one or more processors can convert the received input signals into output signals using sound settings that are based on an acoustic environment associated with a recipient of the hearing device. In addition, the one or more processors can transition to different sound settings based on a change in the recipient's acoustic environment, based on a schedule associated with the recipient, etc.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted above, presented herein are techniques for incrementally adjusting parameters of a medical device to ensure a “smooth” switch or transition between different sets of processing settings/stimulation strategies of the medical device. As used herein, a “smooth” transition is a transition that mitigates perceptual disruption to the recipient (e.g., a transition that is substantially non-perceptible).

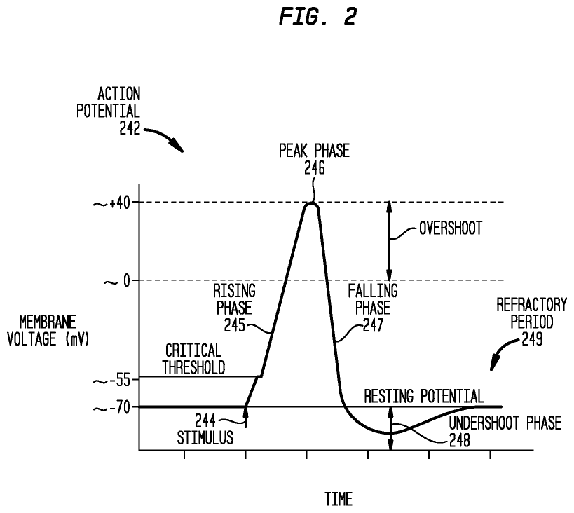

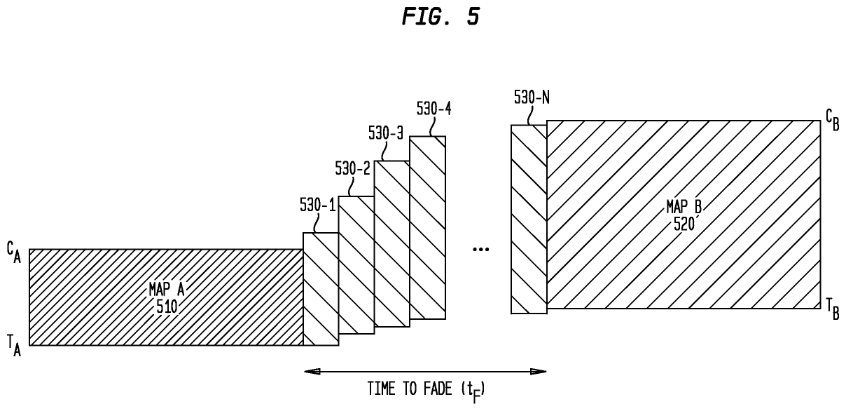

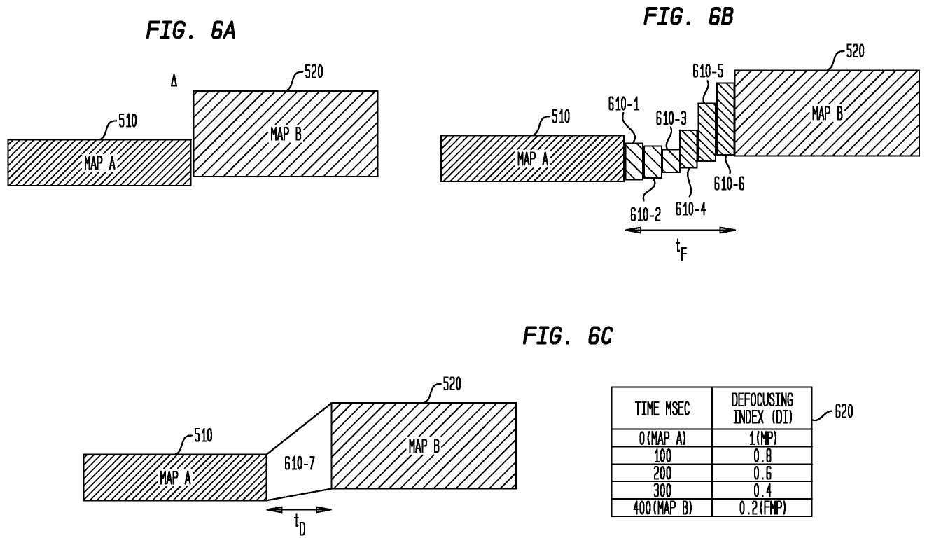

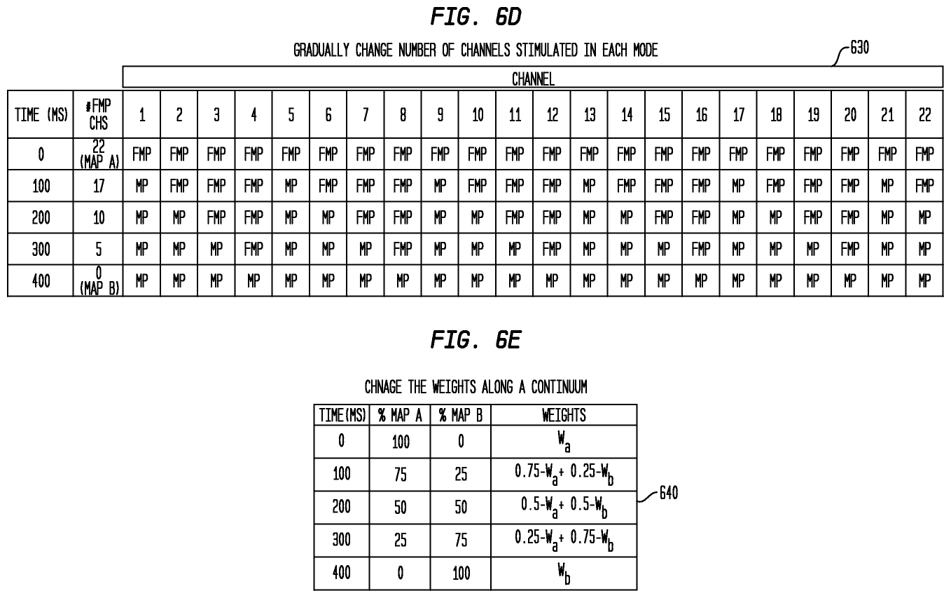

As noted above, one type of transition that can be smoothed using the techniques presented herein is the transition between different stimulation strategies (e.g., different manners in which stimulation is delivered to a recipient). FIGS. 2, 3, and 4A-4D generally illustrate aspects of different stimulation strategies in the context of a cochlear implant, while FIGS. 5 and 6A-6E illustrate further details for smooth transitions, in accordance with embodiments presented herein.

Referring first to FIG. 2, shown are various phases of an idealized action potential 242 as a potential passes through a nerve cell. The action potential is presented as membrane voltage in millivolts (mV) versus time. It is well understood that the human auditory system is composed of many structural components, some of which are connected extensively by bundles of nerve cells (neurons). Each nerve cell has a cell membrane which acts as a barrier to prevent intercellular fluid from mixing with extracellular fluid. The intercellular and extracellular fluids have different concentrations of ions, which leads to a difference in charge between the fluids. This difference in charge across the cell membrane is referred to herein as the membrane potential (Vm) of the nerve cell. Nerve cells use membrane potentials to transmit signals between different parts of the auditory system.

In nerve cells that are at rest (i.e., not transmitting a nerve signal) the membrane potential is referred to as the resting potential of the nerve cell. Upon receipt of a stimulus, the electrical properties of a nerve cell membrane are subjected to abrupt changes, referred to herein as a nerve action potential, or simply action potential. The action potential represents the transient depolarization and repolarization of the nerve cell membrane. The action potential causes electrical signal transmission along the conductive core (axon) of a nerve cell. Signals can be then transmitted along a group of nerve cells via such propagating action potentials.

Returning to FIG. 2, the illustrate membrane voltages and times are for illustration purposes only and the actual values can vary depending on the individual. Prior to application of a stimulus 244 to the nerve cell, the resting potential of the nerve cell is approximately −70 mV. Stimulus 244 is applied at a first time. In normal hearing, this stimulus is provided by movement of the hair cells of the cochlea. Movement of these hair cells results in the release of neurotransmitter into the synaptic cleft, which in return leads to action potentials in individual auditory nerve fibers. In cochlear implants, the stimulus 244 is an electrical stimulation signal (electrical stimulation).

Following application of stimulus 244, the nerve cell begins to depolarize. Depolarization of the nerve cell refers to the fact that the voltage of the cell becomes more positive following stimulus 244. When the membrane of the nerve cell becomes depolarized beyond the cell's critical threshold, the nerve cell undergoes an action potential. This action potential is sometimes referred to as the “firing” or “activation” of the nerve cell. As used herein, the critical threshold of a nerve cell, group of nerve cells, etc. refers to the threshold level at which the nerve cell, group of nerve cells, etc. will undergo an action potential. In the example illustrated in FIG. 2, the critical threshold level for firing of the nerve cell is approximately −50 mV. The critical threshold and other transitions can be different for various recipients and so the values provided in FIG. 2 are merely illustrative.

The course of the illustrative action potential in the nerve cell can be generally divided into five phases. These five phases are shown in FIG. 2 as a rising phase 245, a peak phase 246, a falling phase 247, an undershoot phase 248, and finally a refractory phase (period) 249. During rising phase 245, the membrane voltage continues to depolarize and the point at which depolarization ceases is shown as peak phase 246. In the example of FIG. 2, at this peak phase 246, the membrane voltage reaches a maximum value of approximately 40 mV.

Following peak phase 246, the action potential undergoes falling phase 247. During falling phase 247, the membrane voltage becomes increasingly more negative, sometimes referred to as hyperpolarization of the nerve cell. This hyperpolarization causes the membrane voltage to temporarily become more negatively charged than when the nerve cell is at rest. This phase is referred to as the undershoot phase 248 of action potential 241. Following this undershoot phase 248, there is a time period during which it is impossible or difficult for the nerve cells to fire. This time period is referred to as the refractory phase (period) 249.

As noted above, the nerve cell must obtain a membrane voltage above a critical threshold before the nerve cell can fire/activate. The number of nerve cells that fire in response to electrical stimulation (current) can affect the “resolution” of the electrical stimulation. As used herein, the resolution of the electrical stimulation or the “stimulus resolution” refers to the amount of acoustic detail (i.e., the spectral and/or temporal detail from the input acoustic sound signal(s)) that is delivered by the electrical stimulation at the implanted electrodes in the cochlea and, in turn, received by the primary auditory neurons (spiral ganglion cells). As described further below, electrical stimulation has a number of characteristics/attributes that control the stimulus resolution. These attributes include for example, the spatial attributes of the electrical stimulation, temporal attributes of the electrical stimulation, frequency attributes of the electrical stimulation, instantaneous spectral bandwidth attributes of the electrical stimulation, etc. The spatial attributes of the electrical stimulation control the width along the frequency axis (i.e., along the basilar membrane) of an area of activated nerve cells in response to delivered stimulation, sometimes referred to herein as the “spatial resolution” of the electrical stimulation. The temporal attributes refer to the temporal coding of the electrical stimulation, such as the pulse rate, sometimes referred to herein as the “temporal resolution” of the electrical stimulation. The frequency attributes refer to the frequency analysis of the acoustic input by the filter bank, for example the number and sharpness of the filters in the filter bank, sometimes referred herein as the “frequency resolution” of the electrical stimulation. The instantaneous spectral bandwidth attributes refer to the proportion of the analyzed spectrum that is delivered via electrical stimulation, such as the number of channels stimulated out of the total number of channels in each stimulation frame.

The spatial resolution of electrical stimulation can be controlled, for example, through the use of different electrode configurations for a given stimulation channel to activate nerve cell regions of different widths. Monopolar stimulation, for instance, is an electrode configuration where for a given stimulation channel the current is “sourced” via one of the intra-cochlea electrodes 144, but the current is “sunk” by an electrode outside of the cochlea, sometimes referred to as the extra-cochlear electrode (ECE) 139 (FIG. 1D). Monopolar stimulation typically exhibits a large degree of current spread (i.e., wide stimulation pattern) and, accordingly, has a low spatial resolution. Other types of electrode configurations, such as bipolar, tripolar, focused multi-polar (FMP), a.k.a. “phased-array” stimulation, etc. typically reduce the size of an excited neural population by “sourcing” the current via one or more of the intra-cochlear electrodes 144, while also “sinking” the current via one or more other proximate intra-cochlear electrodes. Bipolar, tripolar, focused multi-polar and other types of electrode configurations that both source and sink current via intra-cochlear electrodes are generally and collectively referred to herein as “focused” stimulation. Focused stimulation typically exhibits a smaller degree of current spread (i.e., narrow stimulation pattern) when compared to monopolar stimulation and, accordingly, has a higher spatial resolution than monopolar stimulation. Likewise, other types of electrode configurations, such as double electrode mode, virtual channels, wide channels, defocused multi-polar, etc. typically increase the size of an excited neural population by “sourcing” the current via multiple neighboring intra-cochlear electrodes.

The cochlea is tonotopically mapped, that is, partitioned into regions each responsive to sound signals in a particular frequency range. In general, the basal region of the cochlea is responsive to higher frequency sounds, while the more apical regions of the cochlea are responsive to lower frequencies. The tonopotic nature of the cochlea is leveraged in cochlear implants such that specific acoustic frequencies are allocated to the electrodes 144 of the stimulating assembly 116 that are positioned close to the corresponding tonotopic region of the cochlea (i.e., the region of the cochlea that would naturally be stimulated in acoustic hearing by the acoustic frequency). That is, in a cochlear implant, specific frequency bands are each mapped to a set of one or more electrodes that are used to stimulate a selected (target) population of cochlea nerve cells. The frequency bands and associated electrodes form a stimulation channel that delivers stimulation signals to the recipient.

In general, it is desirable for a stimulation channel to stimulate only a narrow region of neurons such that the resulting neural responses from neighboring stimulation channels have minimal overlap. Accordingly, the ideal stimulation strategy in a cochlear implant would use focused stimulation channels to evoke perception of all sound signals at any given time. Such a strategy would, ideally, enable each stimulation channel to stimulate a discrete tonotopic region of the cochlea to better mimic natural hearing and enable better perception of the details of the sound signals. Although focused stimulation generally improves hearing performance, this improved hearing performance comes at the cost of significant increased power consumption, added delays to the processing path, and increased complexity, etc. relative to the use of only monopolar stimulation. Additionally, not all listening situations benefit from the increased fidelity offered by focused stimulation as different listening situations present varying levels of difficulty to cochlear implant recipients. For example, understanding speech in a quiet room is easier than understanding the same speech in a busy restaurant with many competing speakers. Accordingly, recipients benefit more or less from the details of sound presented using increased stimulus resolution in different environments.

In accordance with certain embodiments presented herein, a hearing device is configured to analyze received sound signals to determine the primary or main sound “class” of the sound signals. In general, the sound class provides an indication of the difficulty/complexity of a recipient's listening situation/environment (i.e., the environment in which the device is currently/presently located). Based on the sound class of the sound signals, the hearing device is configured to set the operational (e.g., sound processing) settings of the electrical stimulation signals that are delivered to the recipient to evoke perception of the sound signals. The operational settings are set in a manner that optimizes the tradeoff between hearing performance (e.g., increased fidelity) and power consumption (e.g., battery life). The hearing device uses higher resolution stimulation (i.e., stimulation that provides relatively more acoustic detail) in more challenging listening situations with increased expected listening effort, and uses lower resolution stimulation (i.e., stimulation that provides relatively less acoustic detail) in easier listening situations with lower expected listening effort. Since there is limited power available in a cochlear implant, it is therefore advantageous to adapt the stimulation resolution depending on the listening situation in order to optimize the stimulus resolution for the best overall hearing performance within the long-term power budget.

When a recipient experiences a change to the acoustic environment (e.g., travels to a new environment with different background noise), the hearing device can determine the primary or main sound “class” of the new sound signals and determine a new set of operational (e.g., sound processing) settings for use in generating and/or delivering stimulation signals (e.g., electrical stimulating signals) to the recipient. Abruptly changing from a first set of operational settings to a second set of operational settings can cause a perceptual disruption to a recipient of the hearing device. In accordance with the embodiments presented herein, the stimulation parameters, that is the parameters associated with the processing and/or delivery of the stimulation signals by the hearing device, can be adjusted in an incremental (e.g., stepwise or gradual) manner to create a smooth transition between the first set of operational settings to the second set of operational settings. The incrementally adjustable parameters can include, for example, threshold levels, comfort levels, volume levels, degree of focusing, a number of channels stimulated in different electrode configurations, electrode weights, and/or different parameters. In some embodiments, incrementally adjusting parameters may include switching between sound coding strategies. For example, switching between a sound coding strategy that requires power at a higher rate (e.g., an advanced combination encoder (ACE) strategy) to a more battery efficient sound coding strategy at a lower rate (e.g., a Fundamental Asynchronous Stimulus Timing (FAST) strategy) may increase battery autonomy.

In accordance with the embodiments presented herein, the degree of focusing can be adjusted via a spatial resolution change (e.g., adjusting the spatial attributes of the electrical stimulation). For example, the spatial resolution can be increased through use of a more focused stimulation strategy. Conversely, the spatial resolution can be lowered, for example, through the use of monopolar stimulation or a wider/defocused stimulation strategy. These decreases in the spatial resolution have the benefit of lower power consumption and lower complexity, but they also sacrifice listening fidelity (e.g., loss of sound details).

In addition, the temporal resolution (i.e., the temporal attributes of the electrical stimulation) can be varied, for example, by changing the rate of the current pulses forming the electrical stimulation. Higher pulse rates offer higher temporal resolution and use more power, while lower pulse rates offer lower temporal resolution and are more power efficient. As noted above, the term stimulus resolution can refer to both the spatial resolution and the temporal resolution, as well as other attributes (e.g., frequency attributes of the electrical stimulation, instantaneous spectral bandwidth attributes of the electrical stimulation, etc.). As such, reference to a change in stimulation resolution can refer one a change in any of the above attributes related to the stimulus resolution.

In general, the stimulus resolution can be varied with differing associated power costs and, in certain situations, the techniques presented herein purposely downgrade hearing performance (e.g., speech perception) to reduce power consumption. However, this downgrade in hearing performance is dynamically activated only in listening situations where the recipient likely does not have difficulty understanding/perceiving the sound signals with lower stimulus resolution (e.g., monopolar stimulation, defocused stimulation, etc.) and/or does not need the details provided by high resolution (e.g., focused stimulation). A downgrade in hearing performance may additionally be activated in environments of pure noise (e.g., environments without speech) and pure quiet without affecting a recipient's listening experience.

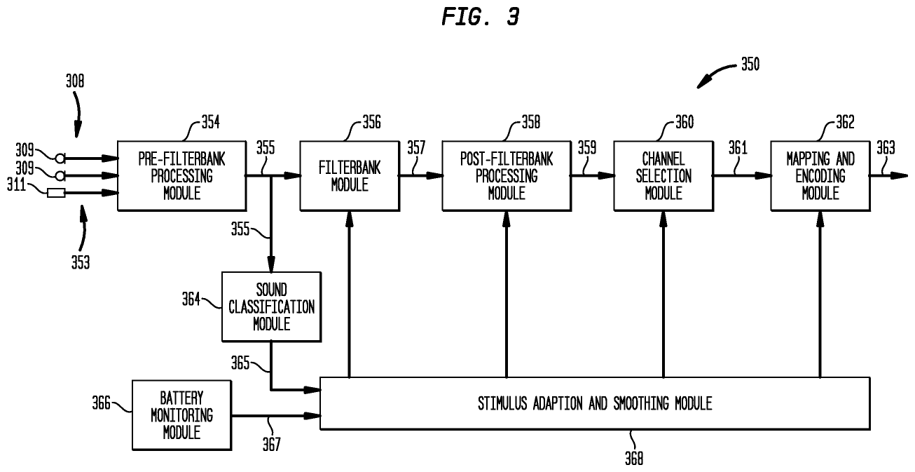

FIG. 3 is a schematic diagram illustrating the general signal processing path 350 of a cochlear implant, such as cochlear implant 102, in accordance with embodiments presented herein. As noted, the cochlear implant 102 comprises one or more sound input elements 308. In the example of FIG. 3, the sound input elements 308 comprise two microphones 309 and at least one auxiliary input 311 (e.g., an audio input port, a cable port, a telecoil, a wireless transceiver, etc.). If not already in an electrical form, sound input elements 308 convert received/input sound signals into electrical signals 353, referred to herein as electrical input signals, that represent the received sound signals. As shown in FIG. 3, the electrical input signals 353 are provided to a pre-filterbank processing module 354.

The pre-filterbank processing module 354 is configured to, as needed, combine the electrical input signals 353 received from the sound input elements 308 and prepare those signals for subsequent processing. The pre-filterbank processing module 354 then generates a pre-filtered output signal 355 that, as described further below, is the basis of further processing operations. The pre-filtered output signal 355 represents the collective sound signals received at the sound input elements 308 at a given point in time.

The cochlear implant 102 is generally configured to execute sound processing and coding to convert the pre-filtered output signal 355 into output signals that represent electrical stimulation for delivery to the recipient. As such, the sound processing path 350 comprises a filterbank module (filterbank) 356, a post-filterbank processing module 358, a channel selection module 360, and a channel mapping and encoding module 362.

In operation, the pre-filtered output signal 355 generated by the pre-filterbank processing module 354 is provided to the filterbank module 356. The filterbank module 356 generates a suitable set of bandwidth limited channels, or frequency bins, that each includes a spectral component of the received sound signals. That is, the filterbank module 356 comprises a plurality of band-pass filters that separate the pre-filtered output signal 355 into multiple components/channels, each one carrying a single frequency sub-band of the original signal (i.e., frequency components of the received sounds signal).

The channels created by the filterbank module 356 are sometimes referred to herein as sound processing channels, and the sound signal components within each of the sound processing channels are sometimes referred to herein as band-pass filtered signals or channelized signals. The band-pass filtered or channelized signals created by the filterbank module 356 are processed (e.g., modified/adjusted) as they pass through the sound processing path 350. As such, the band-pass filtered or channelized signals are referred to differently at different stages of the sound processing path 350. However, it will be appreciated that reference herein to a band-pass filtered signal or a channelized signal can refer to the spectral component of the received sound signals at any point within the sound processing path 350 (e.g., pre-processed, processed, selected, etc.).

At the output of the filterbank module 356, the channelized signals are initially referred to herein as pre-processed signals 357. The number ‘m’ of channels and pre-processed signals 357 generated by the filterbank module 356 can depend on a number of different factors including, but not limited to, implant design, number of active electrodes, coding strategy, and/or recipient preference(s). In certain arrangements, twenty-two (22) channelized signals are created and the sound processing path 350 is said to include 22 channels.

The pre-processed signals 357 are provided to the post-filterbank processing module 358. The post-filterbank processing module 358 is configured to perform a number of sound processing operations on the pre-processed signals 357. These sound processing operations include, for example, channelized gain adjustments for hearing loss compensation (e.g., gain adjustments to one or more discrete frequency ranges of the sound signals), noise reduction operations, speech enhancement operations, etc., in one or more of the channels. After performing the sound processing operations, the post-filterbank processing module 358 outputs a plurality of processed channelized signals 359.

In the specific arrangement of FIG. 3, the sound processing path 350 includes a channel selection module 360. The channel selection module 360 is configured to perform a channel selection process to select, according to one or more selection rules, which of the ‘m’ channels should be use in hearing compensation. The signals selected at channel selection module 360 are represented in FIG. 3 by arrow 361 and are referred to herein as selected channelized signals or, more simply, selected signals.

In the embodiment of FIG. 3, the channel selection module 360 selects a subset ‘n’ of the ‘m’ processed channelized signals 359 for use in generation of electrical stimulation for delivery to a recipient (i.e., the sound processing channels are reduced from ‘m’ channels to ‘n’ channels). In one specific example, the ‘n’ largest amplitude channels (maxima) from the ‘m’ available combined channel signals/masker signals is made, with ‘m’ and ‘n’ being programmable during initial fitting, and/or operation of the hearing device. It is to be appreciated that different channel selection methods could be used, and are not limited to maxima selection.

It is also to be appreciated that, in certain embodiments, the channel selection module 360 can be omitted. For example, certain arrangements can use a continuous interleaved sampling (CIS), CIS-based, or other non-channel selection sound coding strategy.

The sound processing path 350 also comprises the channel mapping and encoding module 362. The channel mapping and encoding module 362 is configured to map the amplitudes of the selected signals 361 (or the processed channelized signals 359 in embodiments that do not include channel selection) into a set of output signals (e.g., stimulation commands) that represent the attributes of the electrical stimulation signals that are to be delivered to the recipient so as to evoke perception of at least a portion of the received sound signals. This channel mapping can include, for example, threshold and comfort level mapping, dynamic range adjustments (e.g., compression), volume adjustments, etc., and can encompass selection of various sequential and/or simultaneous stimulation strategies.

In the embodiment of FIG. 3, the set of stimulation commands that represent the electrical stimulation signals are encoded for transcutaneous transmission (e.g., via an RF link) to an implantable component 112. This encoding is performed, in the specific example of FIG. 3, at the channel mapping and encoding module 362. As such, channel mapping and encoding module 362 is sometimes referred to herein as a channel mapping and encoding module and operates as an output block configured to convert the plurality of channelized signals into a plurality of output signals 363.

Also shown in FIG. 3 are a sound classification module 364, a battery monitoring module 366, and a stimulus adaption and smoothing module 368. The sound classification module 364 is configured to evaluate/analyze the input sound signals and determine the sound class of the sound signals. That is, the sound classification module 364 is configured to use the received sound signals to “classify” the ambient sound environment and/or the sound signals into one or more sound categories (i.e., determine the input signal type). The sound classes/categories can include, but are not limited to, “Speech,” “Noise,” “Speech+Noise,” “Music,” and “Quiet.” As described further below, the sound classification module 364 can also estimate the signal-to-noise ratio (SNR) of the sound signals. In one example, the operations of the sound classification module 364 are performed using the pre-filtered output signal 355 generated by the pre-filterbank processing module 354.

The sound classification module 364 generates sound classification information/data 365 that is provided to the stimulus adaption and smoothing module 368. The sound classification data 365 represents the sound class of the sound signals and, in certain examples, the SNR of the sound signals. Based on the sound classification data 365, the stimulus adaption and smoothing module 368 is configured to determine a level of stimulus resolution that should be used in delivering electrical stimulation signals to represent (evoke perception of) the sound signals. The level of stimulus resolution that should be used in delivering electrical stimulation signals is sometimes referred to herein as the “target” stimulus resolution.

The stimulus adaption and smoothing module 368 is configured to adjust one or more operations performed in the sound processing path 350 so as to achieve the target stimulus resolution (i.e., adapt the stimulus resolution of the electrical stimulation that is delivered to the recipient). In addition, the stimulus adaption and smoothing module 368 is make the adjustments in a smooth manner so as to mitigate perceptual disruptions to the recipient. For example, the stimulus adaption and smoothing module 368 can adjust operations of the filterbank module 356, the post-filterbank processing module 358, the channel selection module 360, and/or the mapping and encoding module 362 to generate output signals representative of electrical stimulation signals having the target stimulus resolution.

In accordance with embodiments presented herein, the stimulus adaption and smoothing module 368 can adjust operations of the sound processing path 350 at a number of different time scales. For example, the stimulus adaption and smoothing module 368 can determine the target stimulus resolution and make corresponding processing adjustments in response to a triggering event, such as the detection of a change in the listening environment (e.g., when the sound classification data 365 indicates the cochlear implant 102 is in a listening environment that is different from the previous listening environment). Alternatively, the stimulus adaption and smoothing module 368 can determine the target stimulus resolution and make corresponding processing adjusts substantially continuously, periodically (e.g., every 1 second, every 5 seconds, etc.,), etc. According to embodiments described herein, the stimulus adaption and smoothing module 268 can be configured to transition between operational settings (e.g., different sound processing settings) by gradually adjusting parameters to mitigate a perceptual disruption to a recipient.

FIG. 3 illustrates an arrangement in which the cochlear implant 102 also comprises a battery monitoring module 366. The battery monitoring module 366 is configured to monitor the charge status of the battery/batteries (e.g., monitor charge level, remaining battery life, etc.) and provide battery information 367 to the stimulus adaption and smoothing module 368. In addition to the sound classification data 365, the stimulus adaption and smoothing module 368 can also use the battery information 367 to determine the target stimulus resolution and make corresponding processing adjusts to the sound processing path operations. For example, if the battery information 167 indicates that the cochlear implant battery/batteries are below a threshold charge level (e.g., below 20% charge), the stimulus adaption and smoothing module 368 can switch the sound processing path 350 to a power saving mode that uses lower resolution (e.g., monopolar stimulation or defocused stimulation only) to conserve power.

FIG. 3 also illustrates a specific arrangement that includes one sound classification module 364. It is to be appreciated that alternative embodiments can make use of multiple sound classification modules. In such embodiments, the stimulus adaption and smoothing module 368 is configured to utilize the information from each of the multiple sound classification modules to determine a target stimulus resolution and adapt the sound processing operations accordingly (i.e., so that the resulting stimulation has a resolution that corresponds to the target stimulus resolution).

Although FIG. 3 illustrates a cochlear implant arrangement, it is to be appreciated that the embodiments presented herein can also be implemented in other types of medical devices, such as other types of hearing devices. For example, the techniques presented herein can be used in electro-acoustic hearing devices that are configured to deliver both acoustical stimulation and electrical stimulation to a recipient. In such embodiments, the device would include two parallel sound processing paths, where the first sound processing path is an electric sound processing path (cochlear implant sound processing path) similar to that is shown in FIG. 3. In such arrangements, the second sound processing path is an acoustic sound processing path (hearing aid sound processing path) that is configured to generate output signals for use in acoustically stimulating the recipient.

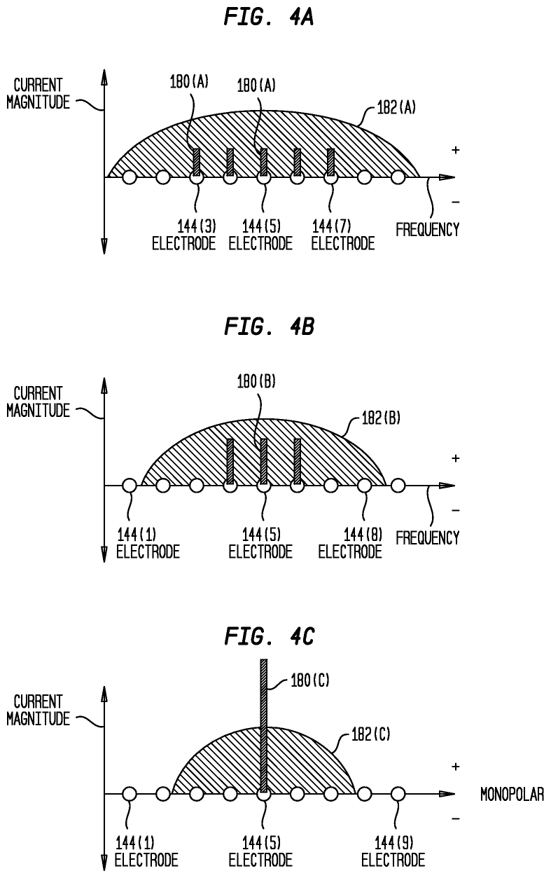

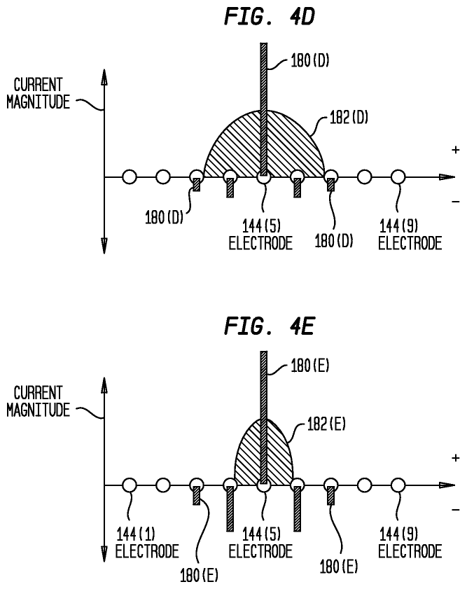

In one embodiment, the operational settings (e.g., sound processing settings) can be switched between a first set of operational settings and a second set of operational settings by switching between different channel/electrode configurations, such as between monopolar stimulation, wide/defocused stimulation, focused (e.g., multipolar current focusing) stimulation, etc. FIGS. 4A-4E are a series of schematic diagrams illustrating exemplary electrode currents and stimulation patterns for five (5) different channel configurations. It is to be appreciated that the stimulation patterns shown in FIGS. 4A-4C are generally illustrative and that, in practice, the stimulation current can spread differently in different recipients.

Each of the FIGS. 4A-4E illustrates a plurality of electrodes shown as electrodes 144(1)-144(9), which are spaced along the recipient's cochlea frequency axis (i.e., along the basilar membrane). FIGS. 4A-4E also include solid lines of varying lengths that extend from various electrodes to generally illustrate the intra-cochlear stimulation current 180(A)-180(E) delivered in accordance with a particular channel configuration. However, it is to be appreciated that stimulation is delivered to a recipient using charge-balanced waveforms, such as biphasic current pulses and that the length of the solid lines extending from the electrodes in each of FIGS. 4A-4E illustrates the relative “weights” that are applied to both phases of the charge-balanced waveform at the corresponding electrode in accordance with different channel configurations. As described further below, the different stimulation currents 180(A)-180(E) (i.e., different channel weightings) results in different stimulation patterns 182(A)-182(E), respectively, of voltage and neural excitation along the frequency axis of the cochlea.

Referring first to FIG. 4C, shown is the use of a monopolar channel configuration where all of the intra-cochlear stimulation current 180(C) is delivered with the same polarity via a single electrode 144(5). In this embodiment, the stimulation current 180(C) is sunk by an extra-cochlear return contact which, for ease of illustration, has been omitted from FIG. 4C. The intra-cochlear stimulation current 180(C) generates a stimulation pattern 182(C) which, as shown, spreads across neighboring electrodes 144(3), 144(4), 144(6), and 144(7). The stimulation pattern 182(C) represents the spatial attributes (spatial resolution) of the monopolar channel configuration.

FIGS. 4A and 4B illustrate wide or defocused channel configurations where the stimulation current is split amongst an increasing number of intracochlear electrodes and, accordingly, the width of the stimulation patterns increases and thus provide increasingly lower spatial resolutions. In these embodiments, the stimulation current 180(A) and 180(B) is again sunk by an extra-cochlear return contact which, for ease of illustration, has been omitted from FIGS. 4A and 4B.

More specifically, in FIG. 4B the stimulation current 180(B) is delivered via three electrodes, namely electrodes 144(4), 144(5), and 144(6). The intra-cochlear stimulation current 180(B) generates a stimulation pattern 182(B) which, as shown, spreads across electrodes 144(2)-144(8). In FIG. 4A, the stimulation current 180(A) is delivered via five electrodes, namely electrodes 144(3)-144(7). The intra-cochlear stimulation current 180(A) generates a stimulation pattern 182(A) which, as shown, spreads across electrodes 144(1)-144(9). In general, the greater the number of nearby electrodes with weights of the same polarity, the lower the spatial resolution of the stimulation signals.

FIGS. 4D and 4E illustrate focused channel configurations where intracochlear compensation currents are added to decrease the spread of current along the frequency axis of the cochlea. The compensation currents are delivered with a polarity that is opposite to that of a primary/main current. In general, the more compensation current at nearby electrodes, the more focused the resulting stimulation pattern (i.e., the lower the width of the stimulus patterns increase and thus increasingly higher spatial resolutions). That is, the spatial resolution is increased by introducing increasing large compensation currents on electrodes surrounding the central electrode with the positive current.

More specifically, in FIG. 4D positive stimulation current 180(D) is delivered via electrode 144(5) and stimulation current 180(D) of opposite polarity is delivered via the neighboring electrodes, namely electrodes 144(3), 144(4), 144(6), and 144(7). The intra-cochlear stimulation current 180(D) generates a stimulation pattern 182(D) which, as shown, only spreads across electrodes 144(4)-144(6). In FIG. 4E, positive stimulation current 180(E) is delivered via electrode 144(5), while stimulation current 180(E) of opposite polarity is delivered via the neighboring electrodes, namely electrodes 144(3), 144(4), 144(6), and 144(7). The intra-cochlear stimulation current 180(E) generates a stimulation pattern 182(E) which, as shown, is generally localized to the spatial area adjacent electrode 144(5).

The difference in the stimulation patterns 182(D) and 182(E) in FIGS. 4D and 4E, respectively, is due to the magnitudes (i.e., weighting) of opposite polarity current delivered via the neighboring electrodes 144(3), 144(4), 144(6), and 144(7). In particular, FIG. 4D illustrates a partially focused configuration where the compensation currents do not fully cancel out the main current on the central electrode and the remaining current goes to a far-field extracochlear electrode (not shown). FIG. 4E is a fully focused configuration where the compensation currents fully cancel out the main current on the central electrode 144(5) (i.e., no far-field extracochlear electrode is needed).

As noted, FIGS. 4A-4E collectively illustrate techniques for adjusting the spatial resolution (i.e., adjusting the spatial attributes of the electrical stimulation) in accordance with embodiments presented herein. However, also as noted, it is to be appreciated that other methods for altering the stimulus resolution could be used in combination with, or as an alternative to, adjustments to the spatial resolution enabled by different stimulation strategies. For example, another technique for adapting the stimulus resolution includes varying the temporal resolution via pulse rate (i.e., higher pulse rates for higher temporal resolutions and lower pulse rates for lower temporal resolutions). In general, changes to the temporal resolution can be implemented in the post-filter bank processing module 358 (e.g., during calculation of the channel envelope signals) and/or in the mapping and encoding module 362 (e.g., selection of the pulse rate).

FIG. 5 illustrates transitioning from a first set of operational settings (e.g., sound processing settings) to a second set of operational settings by gradually adjusting parameters to deliver signals to a recipient using a number of transitional sets of operational settings. FIG. 5 includes map A 510, map B 520, and transitional maps 530-1 to 530-N. As used herein, the term “map” refers to a set of operational settings used to deliver electrical stimulation signals to a recipient of a hearing device, such as cochlear implant system 102, via one or more stimulation channels.

In the example illustrated in FIG. 5, a recipient can be in a first acoustic environment and cochlear implant 102 can be delivering electrical stimulation signals to the recipient using operational settings of map A 510. Map A 510 has a threshold level for each channel, defined collectively as TA, and a comfort levels for each channel, defined collectively as CA. Cochlear implant system 102 can determine to switch the operational settings to the operational settings of map B 520, which has a threshold level for each channel, defined collectively as TB, and a comfort level for each channel, defined collectively as CB.

In one embodiment, the determination to switch from map A 510 to map B 520 can be based on a change in the acoustic environment. For example, the recipient can move from one environment to another environment with a different background noise, such as from a quiet environment to a noisy environment with people talking or with music playing. In this case, cochlear implant system 102 can determine to switch from map A 510 to map B 520 to provide the recipient with a mode of stimulation better adapted to the acoustic environment.

In another embodiment, cochlear implant system 102 can determine to switch from map A 510 to map B 520 at a particular time according to the recipient's schedule. For example, a schedule of switching between operational settings can be generated based on training data associated with a recipient's regular schedule. If the recipient regularly changes acoustic environments at a set time, cochlear implant system 102 can switch from map A 510 to map B 520 at a time indicated by the training data.

Switching from map A 510 to map B 520 without a transition or smoothing/fading can create a perceptual disruption to the recipient that the recipient can find jarring. To mitigate the disruption, parameters associated with map A 510 can be gradually adjusted until the operational settings associated with map B 520 are achieved. As illustrated in FIG. 5, an amount of time to switch between map A 510 and map B 520 is defined by the time to fade (t_{F}). The time to fade can be configurable based on, for example, sound settings associated with map A 510 and/or map B 520, recipient preferences, etc.

In the example illustrated in FIG. 5, parameters associated with map A 510 are adjusted in a stepwise manner. For example, parameters associated with map A 510 are adjusted slightly, which results in transitional map 530-1. After the operational settings are switched from the settings associated with map A 510 to transitional map 530-1, electrical stimulation signals are delivered to the recipient using operational settings of transitional map 530-1. Because the parameters associated with transitional map 530-1 are similar to the parameters associated with map A 510, switching between map A 510 and transitional map 530-1 can produce a minimal or no disruption to a recipient of a hearing device. Parameters associated with transitional map 530-1 can be adjusted slightly, which results in transitional map 530-2, and electrical stimulation signals are delivered to the recipient using operational settings of transitional map 530-2. Once again, switching between transitional map 530-1 and transitional map 530-2 can produce a minimal or no disruption to the recipient. Parameters can continue to be adjusted slightly, resulting in transitional maps 530-3, 530-4, . . . , 530-N, and map B 520.

A number of transitional maps 530-1 to 530-N can depend on a number of factors, such as a desired time to fade, a difference between the sound settings of map A 510 and map B 520, or additional factors. As discussed below with respect to FIGS. 6A-6E, several different parameters or a combination of parameters can be adjusted in a stepwise or fading manner to create a smooth transition between map A 510 and map B 520. For example, the parameters that define each transitional map include Threshold (T) and Loud But Comfortable (C) levels for each channel, a dynamic range (C-T), volume (V), and intracochlear electrode weights (W) to be applied to each stimulating electrode for each map channel. By making multiple smaller changes to parameters associated with operational settings when switching between sets of operational settings, the perceptual disruption resulting from a switch between map A 510 and map B 520 can be mitigated.

FIGS. 6A-6C illustrate example switching modes for switching between map A 510 and map B 520. FIG. 6A illustrates an example in which operational settings are switched from map A 510 to map B 520 without a smoothing of the transition. Switching without smoothing the transition can allow for an understanding of the percept that is driving the A or the change between the two operational settings.

FIG. 6B illustrates an example in which the transition is smoothed using level and/or volume changes. To smooth the transition between map A 510 and map B 520, the loudness is gradually changed by adjusting the T and/or C levels and/or the volume. FIG. 6B illustrates six transitional maps, transitional maps 610-1 to 610-6. Each transitional map is the result of adjusting the T and/or C levels and/or the volume from the previous map. In this example, maps 610-1 to 610-3 have the same weights as map A 510 and maps 610-4 to 610-6 have the weights of map B 520. Because FIG. 6B illustrates six transitional maps, each transitional map is in use for a duration of t_{F}/6. Although FIG. 6B illustrates six transitional maps, a number of transitional maps used during the transition can vary based on a desired time to fade, a difference in operational settings associated with map A 510 and map B 520, recipient preferences, etc.

FIG. 6C illustrates an example in which the transition is smoothed or faded using a gradual change in degree of focusing. FIG. 6C includes map A 510, map B 520, transitional map 610-7, and a table 620. Map A 510, map B 520, and transitional map 610-7 will be referenced with regard to FIGS. 6D and 6E. A defocusing index (DI) indicates a degree of focusing for focused multipolar stimulation. For example, a DI of 1 can correspond to monopolar (MP) stimulation and a DI of 0.2 can correspond to focused multipolar (FMP) stimulation. In the example illustrated in FIG. 6C, map A 510 corresponds to sound settings associated with MP stimulation and map B 520 corresponds to sound settings associated with FMP stimulation. In this example, transition smoothing is performed by gradually changing the DI from map A 510 to map B 520 in transitional map 610-7 over a period of time t_{D}. In this example, to is 400 milliseconds.

As illustrated in table 620, at time 0, the DI is 1. At this point, the operational settings correspond to the set of operational settings associated with map A 510, which corresponds to monopolar stimulation. During time t_{D} (in this case, 400 milliseconds), the DI is gradually changed in the transitional map 610-7. For example, as illustrated in table 620, at time 100 milliseconds, the DI is changed to 0.8. At time 200 milliseconds, the DI is changed to 0.6. At time 300 milliseconds, the DI is changed to 0.4. At time 400 milliseconds, the DI is 0.2, which corresponds to the DI of FMP stimulation associated with map B 520. Although the parameters in this example are changed every 100 milliseconds over a 400 millisecond period of time t_{D}, the length of t_{D} and each interval are exemplary and can be different lengths of time, can change based on the situation, and/or can be customized.

As the DI is gradually changed from map A 510 to map B 520, the T and C levels for the intermediate DIs can also change. The T and C levels can be interpolated between the endpoints corresponding to map A 510 or map B 520, measured directly, estimated from population data, or otherwise inferred.

FIG. 6D illustrates an example in which the transition is smoothed or faded using a gradual change in a number of channels stimulated in different modes or electrode configurations. FIG. 6D includes a table 630 illustrating the channels that are stimulated in each electrode configuration over a period of time t_{D} in transition map 610-7 (illustrated in FIG. 6C). In the example shown in FIG. 6D, map A 510 corresponds to sound settings associated with FMP stimulation, map B 520 corresponds to sound settings associated with MP stimulation, and to is 400 milliseconds.

As shown in table 630, at time zero (0) milliseconds, the sound settings correspond to the set of sound settings associated with map A 510. At this time, each of the 22 channels is stimulated using FMP stimulation. In transition map 610-7, the parameters are gradually changed so that, at time 100 seconds, 17 of the 22 channels are stimulated using FMP stimulation and 5 of the 22 channels are stimulated using MP stimulation. At time 200 milliseconds, the parameters are gradually changed again so that ten (10) of the channels are stimulated using FMP stimulation and 12 of the channels are stimulated using MP stimulation. At time 300 milliseconds, the parameters are gradually changed again so that five (5) of the channels are stimulated using FMP stimulation and 17 of the channels are stimulated using MP stimulation. At time 400 milliseconds, all 22 of the channels are stimulated using MP stimulation, which corresponds to the sound settings of map B 520. At this point, transitional map 610-7 ends and the electrical stimulation signals are delivered to the recipient using the sound settings of map B 520. Although the parameters in this example are changed every 100 milliseconds over a 400 millisecond period of time t_{D}, the length of t_{D} and each interval are exemplary and can be different lengths of time, can change based on the situation, and/or can be customized.

In the example illustrated in FIG. 6D, the T levels and C levels for each channel are either the T and C levels of map A 510 or map B 520. The T levels and C levels are not ramped in transitional map 610-7.

FIG. 6E illustrates an example in which the transition is smoothed or faded by changing channel weights along a continuum. In this example, the weights for each transitional map are a weighted average or some other combination of the weights of map A 510 and map B 520. FIG. 6E includes table 640, which illustrates gradually changing the weights applied to electrodes over a 400 millisecond period of time (t_{D}) of transitional map 610-7 (illustrated in FIG. 6C).

As shown in table 640, at time zero (0) milliseconds, the weights are the weights of map A 510 (Wₐ) and zero (0) percent the weights of map B 520 (W_{b}). At time 100 milliseconds, the weights are gradually changed so the weights are 75% Wₐ and 25% W_{b} (weights=0.75·Wₐ+0.25·W_{b}). At time 200 milliseconds, the weights are 50% Wₐ and 50% W_{b} (weights=0.5·Wₐ+0.5·W_{b}). At time 300 milliseconds, the weights are 25% Wₐ and 75% W_{b} (weights=0.25·Wₐ+0.25·W_{b}). At time 400 milliseconds, the weights are 100% W_{b} and 0% Wₐ, which corresponds to the parameters of map B 520. Although the parameters in this example are changed every 100 milliseconds over a 400 millisecond period of time t_{D}, the length of t_{D} and each interval are exemplary and can be different lengths of time, can change based on the situation, and/or can be customized, including by applying different weighting tables for different stimulation channels.

In the example illustrated in FIG. 6E, the T levels and C levels for transitional maps 610-7 can be interpolated between the endpoints corresponding to map A 510 and map B 520, measured directly, estimated from population data, or otherwise inferred.

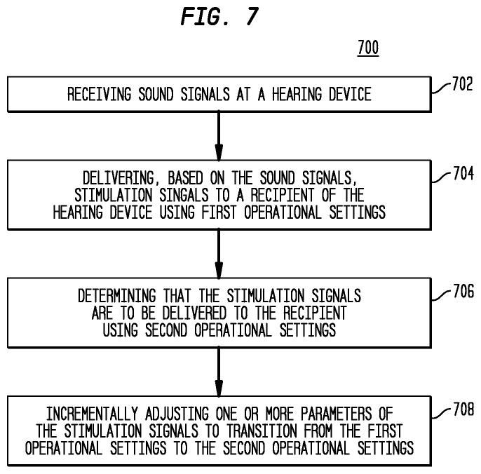

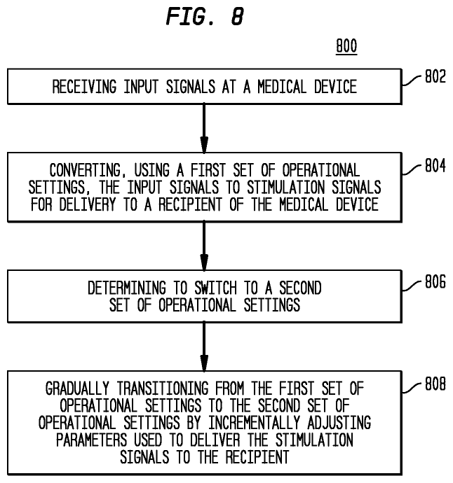

FIG. 7 is a flowchart illustrating a method 700 in accordance with embodiments presented herein. Method 700 begins at 702 where a hearing device receives sound signals. At 704, the hearing device delivers, based on the sound signals, stimulation signals (e.g., acoustic stimulation signals, mechanical stimulation signals, and/or electrical stimulation signals) to a recipient of the hearing device using first operational settings. At 706, the hearing device determines that the stimulation signals are to be delivered to the recipient using second operational settings. At 708, the hearing device incrementally adjusts one or more parameters of the stimulation signals to transition from the first operational settings to the second operational settings. Incrementally adjusting one or more parameters of the stimulation signals to transition from the first operational settings to the second operational settings can refer to adjust parameters used to generate the stimulation signals.

FIG. 8 is a flowchart illustrating a method 800 in accordance with embodiments presented herein. Method 800 begins at 802 where a medica device receives input signals. At 804, the medical device converts, using a first set of operational settings, the input signals to stimulation signals for delivery to a recipient of the medical device. At 806, a determination is made to switch to a second set of operational settings. At 808, the medical device gradually transitions from use of the first set of stimulation signals to use of the second set of operational settings by incrementally adjusting parameters used to deliver the stimulation signals to the recipient.

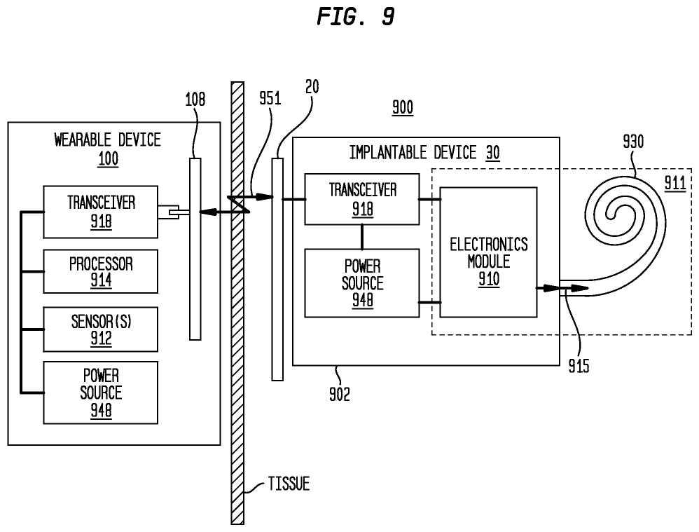

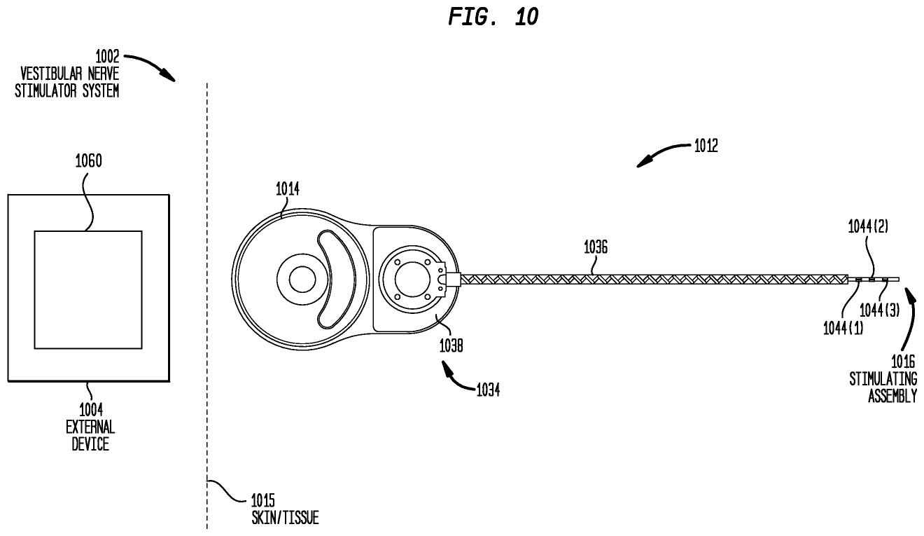

As previously described, the technology disclosed herein can be applied in any of a variety of circumstances and with a variety of different devices. Example devices that can benefit from technology disclosed herein are described in more detail in FIGS. 9 and 10, below. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 9 is a functional block diagram of an implantable stimulator system 900 that can benefit from the technologies described herein. The implantable stimulator system 900 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 902. Here, the wearable device 100 is configured to transcutaneously couple with the implantable device 30 via a wireless connection to provide additional functionality to the implantable device 30.

In the illustrated example, the wearable device 100 includes one or more sensors 912, a processor 914, a transceiver 918, and a power source 948. The one or more sensors 912 can be one or more units configured to produce data based on sensed activities. In an example where the stimulation system 900 is an auditory prosthesis system, the one or more sensors 912 include sound input sensors, such as a microphone, an electrical input for an FM hearing system, other components for receiving sound input, or combinations thereof. Where the stimulation system 900 is a visual prosthesis system, the one or more sensors 912 can include one or more cameras or other visual sensors. Where the stimulation system 900 is a cardiac stimulator, the one or more sensors 912 can include cardiac monitors. The processor 914 can be a component (e.g., a central processing unit) configured to control stimulation provided by the implantable device 30. The stimulation can be controlled based on data from the sensor 912, a stimulation schedule, or other data. Where the stimulation system 900 is an auditory prosthesis, the processor 914 can be configured to convert sound signals received from the sensor(s) 912 (e.g., acting as a sound input unit) into signals 951. The transceiver 918 is configured to send the signals 951 in the form of power signals, data signals, combinations thereof (e.g., by interleaving the signals), or other signals. The transceiver 918 can also be configured to receive power or data. Stimulation signals can be generated by the processor 914 and transmitted, using the transceiver 918, to the implantable device 30 for use in providing stimulation.

In the illustrated example, the implantable device 30 includes a transceiver 918, a power source 948, and a medical instrument 911 that includes an electronics module 910 and a stimulator assembly 930. The implantable device 30 further includes a hermetically sealed, biocompatible implantable housing 902 enclosing one or more of the components.

The electronics module 910 can include one or more other components to provide medical device functionality. In many examples, the electronics module 910 includes one or more components for receiving a signal and converting the signal into the stimulation signal 915. The electronics module 910 can further include a stimulator unit. The electronics module 910 can generate or control delivery of the stimulation signals 915 to the stimulator assembly 930. In examples, the electronics module 910 includes one or more processors (e.g., central processing units or microcontrollers) coupled to memory components (e.g., flash memory) storing instructions that when executed cause performance of an operation. In examples, the electronics module 910 generates and monitors parameters associated with generating and delivering the stimulus (e.g., output voltage, output current, or line impedance). In examples, the electronics module 910 generates a telemetry signal (e.g., a data signal) that includes telemetry data. The electronics module 910 can send the telemetry signal to the wearable device 100 or store the telemetry signal in memory for later use or retrieval.

The stimulator assembly 930 can be a component configured to provide stimulation to target tissue. In the illustrated example, the stimulator assembly 930 is an electrode assembly that includes an array of electrode contacts disposed on a lead. The lead can be disposed proximate tissue to be stimulated. Where the system 900 is a cochlear implant system, the stimulator assembly 930 can be inserted into the recipient's cochlea. The stimulator assembly 930 can be configured to deliver stimulation signals 915 (e.g., electrical stimulation signals) generated by the electronics module 910 to the cochlea to cause the recipient to experience a hearing percept. In other examples, the stimulator assembly 930 is a vibratory actuator disposed inside or outside of a housing of the implantable device 30 and configured to generate vibrations. The vibratory actuator receives the stimulation signals 915 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 918 can be components configured to transcutaneously receive and/or transmit a signal 951 (e.g., a power signal and/or a data signal). The transceiver 918 can be a collection of one or more components that form part of a transcutaneous energy or data transfer system to transfer the signal 951 between the wearable device 100 and the implantable device 30. Various types of signal transfer, such as electromagnetic, capacitive, and inductive transfer, can be used to usably receive or transmit the signal 951. The transceiver 918 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 948 can be one or more components configured to provide operational power to other components. The power source 948 can be or include one or more rechargeable batteries. Power for the batteries can be received from a source and stored in the battery. The power can then be distributed to the other components as needed for operation.

As should be appreciated, while particular components are described in conjunction with FIG. 9, technology disclosed herein can be applied in any of a variety of circumstances. The above discussion is not meant to suggest that the disclosed techniques are only suitable for implementation within systems akin to that illustrated in and described with respect to FIG. 9. In general, additional configurations can be used to practice the methods and systems herein and/or some aspects described can be excluded without departing from the methods and systems disclosed herein.

FIG. 10 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 1060. As such, the external device 1004 is configured to transfer data (and potentially power) to the vestibular stimulator 1012,

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 134 also includes an internal/implantable coil 1014 that is generally external to the housing 1038, but which is connected to the transceiver via a hermetic feedthrough (not shown).

The stimulating assembly 1016 comprises a plurality of electrodes 1044(1)-(3) disposed in a carrier member (e.g., a flexible silicone body). In this specific example, the stimulating assembly 1016 comprises three (3) stimulation electrodes, referred to as stimulation electrodes 1044(1), 1044(2), and 1044(3). The stimulation electrodes 1044(1), 1044(2), and 1044(3) function as an electrical interface for delivery of electrical stimulation signals to the recipient's vestibular system.

The stimulating assembly 1016 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 1012, the external device 1004, and/or another external device, can be configured to implement the techniques presented herein. That is, the vestibular stimulator 1012, possibly in combination with the external device 1004 and/or another external device, can be configured for smooth switching between different settings, such as different stimulation strategies, as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method comprising:
receiving environmental signals at a sensory device;
delivering, based on the environmental signals, stimulation signals to a recipient of the sensory device using first operational settings;
determining that the stimulation signals are to be delivered to the recipient using second operational settings that are different from the first operational settings; and
incrementally adjusting one or more parameters of the stimulation signals to transition from the first operational settings to the second operational settings.

2. The method of claim 1, wherein incrementally adjusting the one or more parameters of the stimulation signals includes incrementally adjusting one or more of a threshold level, a comfort level, or a volume level associated with the stimulation signals.

3. The method of claim 1, wherein incrementally adjusting the one or more parameters of the stimulation signals includes incrementally adjusting a stimulus resolution of the stimulation signals.

4. The method of claim 3, wherein incrementally adjusting the stimulus resolution of the stimulation signals includes incrementally adjusting a spatial resolution of the stimulation signals.

5. The method of claim 3, wherein incrementally adjusting the stimulus resolution of the stimulation signals includes incrementally adjusting a temporal resolution of the stimulation signals.

6. (canceled)

7. The method of claim 1, wherein the first operational settings are associated with a first electrode configuration and the second operational settings are associated with a second electrode configuration.

8. The method of claim 7, wherein the one or more parameters of the stimulation signals includes adjusting a number of stimulation channels that are stimulated in the first electrode configuration and the second electrode configuration.

9. The method of claim 1, wherein incrementally adjusting the one or more parameters of the stimulation signals includes incrementally adjusting electrode weights applied to electrodes of one or more stimulation channels.

10. The method of claim 1, wherein the first operational settings and the second operational settings are sound processing settings.

11. The method of claim 1, wherein incrementally adjusting the one or more parameters includes adjusting at least one of the one or more parameters in a stepwise manner.

12. The method of claim 1, wherein determining that the stimulation signals are to be delivered to the recipient using second operational settings is based on a change in an environmental classification of the environmental signals.

13. The method of claim 1, wherein determining that the stimulation signals are to be delivered to the recipient using second operational settings is based on a schedule associated with the recipient.

14. (canceled)

15. One or more non-transitory computer readable storage media comprising instructions that, when executed by a processor, cause the processor to:
receive input signals at a medical device;
convert, using a first set of operational settings, the input signals to stimulation signals for delivery to a recipient of the medical device;
determine to switch to a second set of operational settings; and
gradually transition from use of the first set of operational settings to use of the second set of operational settings by incrementally adjusting parameters used to deliver the stimulation signals to the recipient.

16. The one or more non-transitory computer readable storage media of claim 15, wherein, when gradually transitioning from use of the first set of operational settings to use of the second set of operational settings, the instructions further cause the processor to incrementally adjust one or more of a threshold level, a comfort level, or a volume level associated with the stimulation signals.

17. The one or more non-transitory computer readable storage media of claim 15, wherein, when gradually transitioning from use of the first set of operational settings to use of the second set of operational settings, the instructions further cause the processor to incrementally adjust a stimulus resolution of the stimulation signals.

18. (canceled)

19. (canceled)

20. (canceled)

21. The one or more non-transitory computer readable storage media of claim 15, wherein the first set of operational settings are associated with a first electrode configuration and the second set of operational settings are associated with a second electrode configuration.

22. The one or more non-transitory computer readable storage media of claim 21, wherein, when gradually transitioning from use of the first set of operational settings to use of the second set of operational settings, the instructions further cause the processor to incrementally adjust a number of stimulation channels that are stimulated in the first electrode configuration and the second electrode configuration.

23. The one or more non-transitory computer readable storage media of claim 15, wherein, when gradually transitioning from use of the first set of operational settings to use of the second set of operational settings, the instructions further cause the processor to incrementally adjust electrode weights applied to electrodes of one or more stimulation channels.

24. (canceled)

25. (canceled)

26. A medical device comprising:
one or more input elements configured to receive input signals;
a processing path configured to convert the input signals into one or more output signals for delivery to a recipient of the medical device using a first set of operational settings; and
a stimulus adaption and smoothing module configured to gradually adjust, over a period of time, operation of the processing path from use of the first set of operational settings to use of a second set of operational settings.

27. (canceled)

28. The medical device of claim 26, wherein the stimulus adaption and smoothing module is configured to incrementally adjust operation of the processing path to incrementally change a stimulus resolution of the output signals.

29. The medical device of claim 28, wherein the stimulus adaption and smoothing module is configured to incrementally adjust operation of the processing path to incrementally change at least one of a spatial resolution of the output signals or a temporal resolution of the output signals.

30. (canceled)

31. The medical device of claim 26, wherein the first set of operational settings are associated with a first electrode configuration and the second set of operational settings are associated with a second electrode configuration, and wherein the stimulus adaption and smoothing module is configured to incrementally adjust a number of stimulation channels that are stimulated in the first electrode configuration and the second electrode configuration.

32. The medical device of claim 26, wherein the stimulus adaption and smoothing module is configured to incrementally adjust electrode weights applied to electrodes of one or more stimulation channels.

33. The medical device of claim 26, wherein the input signals comprise one or more environmental signals, and wherein the stimulus adaption and smoothing module is configured to gradually adjust operation of the processing path based on one or more attributes of the one or more environmental signals.

34. (canceled)

35. The medical device of claim 26, further comprising at least one battery, and wherein the stimulus adaption and smoothing module is configured to gradually adjust operation of the processing path based one or more attributes of the at least one battery.

36. (canceled)
